(19) United States
(12) Patent Application Publication
(10) Pub. No.: US 2014/0038917 A1
(43) Pub. Date: Feb. 6, 2014

(54) STERILE AND INJECTABLE AQUEOUS FORMULATION FOR ADMINISTRATION IN THE INTRA-ARTICULAR SPACE OF AN INTRA-ARTICULAR JOINT

(71) Applicants: Samuel Gavard Molliard, Bogeve (FR); Olivier Benoit, Epagny (FR)

(72) Inventors: Samuel Gavard Molliard, Bogeve (FR); Olivier Benoit, Epagny (FR)

(73) Assignee: ANTEIS S.A., Plan-les-Ouates (CH)

(21) Appl. No.: 14/112,437

(22) PCT Filed: Apr. 19, 2012
(86) PCT No.: PCT/IB2012/051962

§ 371 Date: Oct. 17, 2013

Related U.S. Application Data
(60) Provisional application No. 61/476,843, filed on Apr. 19, 2011.

(51) Int. Cl. A61K 47/10; A61K 31/728

(52) U.S. Cl. CPC A61K 47/10; A61K 31/728
USPC 514/54

(57) ABSTRACT

The present invention relates generally to the treatment and/or prevention of pain associated with an intra-articular joint degeneration or disease. This invention concerns in particular a sterile and injectable aqueous formulation for administration in the intra-articular space of an intra-articular joint of a subject, in the form of a gel.

### TECHNICAL FIELD

The present invention relates generally to the treatment and/or prevention of pain associated with an intra-articular joint degeneration or disease. This invention concerns in particular a sterile and injectable aqueous formulation for administration in the intra-articular space of an intra-articular joint of a subject, in the form of a gel.

### BACKGROUND OF THE INVENTION

Intra-articular corticosteroid injections have long been used to treat intra-articular joint degeneration such as osteoarthritis, whereas intra-articular hyaluronic acid injections for only a few years (Vanderstraeten et al., 2005).

Corticosteroids are usually administered into the joint in one single dose. In contrast to viscosupplementation based on hyaluronic acid performed with either one or several injections, corticosteroids provide rapid relief of pain associated with an intra-articular joint degeneration (generally, reduction of the pain in 1 or 2 weeks). With hyaluronic acid, the improvement is delayed and occurs usually 6 to 8 weeks after the injections but the efficacy is of longer duration than for a corticosteroid injection (more than 6 months).

Intra-articular corticosteroid therapy is essentially palliative and must be considered as adjunctive to other basis therapies (Schumacher and Chen, 2005). This therapy is contra indicated when the subject to be treated is suffering from local or generalized infection, immune deficiency or coagulation disorders. Corticosteroid injections may also have a number of side effects such as, for example, facial flushing, chemical synovitis, septic arthritis and a variety of systemic effects (Vanderstraeten et al., 2005). There are numerous reports of hypersensitivity reactions to corticosteroids described in the literature. For example, in his article, Mace reported an anaphylactic shock after intraarticular injection of a corticosteroid (Mace et al., 1997). Furthermore, the use of corticosteroids in osteoarthritis has been controversial: early studies in mice, rats and rabbits suggested that multiple corticosteroid injections might alter cartilage protein synthesis and consequently damage the cartilage (Celeste et al., 2005).

In contrast, as described by Vanderstraeten, more than 10 million viscosupplementation injections with hyaluronic acid products were administered in the USA in 1998, with local reactions in only 3% of cases. During the same period, 16,500 patients died of side effects of anti-inflammatory drugs for osteoarthritis. No anaphylactic or generalized side effects of intra-articular hyaluronic acid injections have been reported. Adverse reactions have been reduced even more by the introduction of the new generation of biotechnologically produced hyaluronic acid products (Vanderstraeten et al., 2005).

Treatment by viscosupplemention consists in injecting a gel containing as a main ingredient the hyaluronic acid into the joint so as to replace the deficient synovial fluid. The viscosupplementation can lessen or stop the pain and contribute to restoring the mobility of the joint. These gels can be based on hyaluronic acid of animal or non-animal origin and can be cross-linked (the case of Synvisc®, Durolane®) or non-cross-linked (the case of Synocrom®, Arthrum®, Lubravisc®, Structovial®).

The persistence of a hyaluronic-acid-based gel is low in a joint (from several hours to several days) and is explained by degradation (by depolymerization) of the hyaluronic acid. The primary factors of degradation of the hyaluronic acid in the joint are free radical degradation, thermal degradation at 37° C., and mechanical degradation (enzymatic degradation is not a significant factor of degradation in the joint). Although the therapeutic effectiveness of the viscosupplement is of longer duration than its dwell time in the joint, the persistence of a gel based on hyaluronic acid in the joint is a prominent parameter that governs the effectiveness of the product. Thus, the longer the dwell time of the hyaluronic-acid-based gel in the joint, the more effective the viscosupplementation treatment.

Viscosupplementation commonly employs three to five intra-articular injections, which are typically administered weekly (the case of Synvisc® for a dosage regimen of 3 injections a week apart). On the market, some viscosupplements are now proposed for “one-shot” administration (the case of Synvisc® One for a single injection).

Recently, the Applicants of the present application have shown in PCT/FR08/00948 (Anteis SA) that the presence of a polyol in a sterile aqueous formulation that is based on hyaluronic acid makes it possible to increase significantly the resistance to the degradation of this gel in the joint.

However, although the optimization of the various parameters listed above allowed the persistence of a hyaluronic-acid-based gel, it did not permit a rapid and strong reduction of pain after just a single injection (reduction of the pain in 1 or 2 weeks), as obtained with corticosteroids, in order to offer to the practitioners an alternative to the treatment with corticosteroids, which allows a decrease of the pain after only one injection but which presents side effects as post-injection flare, synovitis and potential cartilage damage with repeated use.

### SUMMARY OF THE INVENTION

This object has been achieved by providing a sterile and injectable aqueous formulation for administration in the intra-articular space of an intra-articular joint of a subject, in the form of a gel containing i) hyaluronic acid, or one of its salts, at a concentration comprised between 5 to 45 mg/ml, said hyaluronic acid, or one of its salts, having an average molecular weight equal or higher than 500 000 daltons, and ii) a polyol at a concentration equal or higher than 7 mg/ml, wherein the ratio between the concentrations of polyol and acid hyaluronic, or one of its salts, is comprised between about 0.155 to 14 and wherein said sterile and injectable aqueous formulation is adapted for the administration in one single dose injection and has a zero-shear rate viscosity η0 equal or higher than 15 Pa·s.

A further object of the present invention is to provide a sterile and injectable aqueous formulation for use in the treatment and/or prevention of pain associated with an intra-articular joint degeneration or disease.

Another object is to provide a device comprising a sterile and injectable aqueous formulation for use in the treatment and/or prevention of pain associated with an intra-articular joint degeneration or disease.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The terms “comprise”, “comprised” or “comprising” is generally used in the sense of include/including, that is to say permitting the presence of one or more features or components.

As used in the description and claims, the singular form “a”, “an” and “the” include plural references unless the context clearly dictates otherwise.

As used herein, “at least one” means “one or more.”

“Administration” or “administering”, as it applies in the present invention, refers to contact of a therapeutically effective amount of the sterile and injectable aqueous formulation of the invention, to the subject. Preferably, the present invention contemplates administration surgically or by injection or a combination thereof. Administration can be continuous or intermittent.

The subject is preferably an animal, most preferably a mammal and even more preferably a human.

A “sterile” formulation as it applies in the present invention, is one that is free of viable microbes as determined using the USP sterility test (See “The United States Pharmacopeia”, 30th Revision, The United States Pharmacopeia Convention: 2008″).

“Intra-articular joint” refers e.g. to, ankle, hip, wrist, hand joint, knee, foot joint, spine joint, shoulder joint, any other joint or space, etc.

The term “gel” or “hydrogel” refers to a water-containing three dimensional hydrophilic polymer network or gel in which the water is the continuous phase and in which the water content is greater than 50% (w/w).

So far, there is no alternative therapy to intra-articular corticosteroid injection which permits a rapid and strong reduction of pain. In the case of the use of hyaluronic acid based viscosupplements, a beneficial effect on pain appears only a few weeks after the injections (e.g., after the sixth or eighth week) and lasts from 6 months to 12 months.

Surprisingly, the Applicants of the present invention have shown that a rapid, strong and long lasting reduction of pain can be obtained by administering in only one dose of an appropriate volume of a sterile and injectable aqueous formulation in the intra-articular space of an intra-articular joint of a subject, in the form of a gel, said formulation comprising

i) hyaluronic acid, or one of its salts, at a concentration comprised between 5 to 45 mg/ml, said hyaluronic acid, or one of its salts, having an average molecular weight equal or higher than 500 000 daltons, and

ii) at least one polyol at a concentration equal or higher than 7 mg/ml,

wherein the ratio between the concentrations of the at least one polyol and acid hyaluronic, or one of its salts, is comprised between about 0.155 to 14 and wherein said sterile and injectable aqueous formulation is adapted for the administration in one single dose injection and has a zero-shear rate viscosity η0 equal or higher than 15 Pa·s.

In contrast to treatments with hyaluronic based viscosupplements known in the art, the sterile and injectable aqueous formulation of the invention provides a rapid and strong pain relief in a single injection (reduction of the pain in 1 or 2 weeks/level of reduction of the pain similar to the pain relief obtained with a corticosteroid injection).

The beneficial effect on pain of the sterile and injectable aqueous formulation of the invention appears also to be long lasting (up to 6 months of knee osteoarthritis pain relief with a single injection).

The sterile and injectable aqueous formulation of the invention includes hyaluronic acid. As used herein “hyaluronic acid,” refers to a linear long-chain polysaccharide comprising repeating D-glucuronate and N-acetylglucosamine disaccharide units. It can be obtained, for example, either by extraction from animal tissues, such as rooster combs and umbilical cords (Klein, J., & Meyer, F. A., 1983, Biochem. & Biophys. Acta, 755(3), 400-411), or bacterial fermentation (Van Brunt, J., 1986, Biotechnology, 4, 780-782).

The hyaluronic acid can further be subjected for example to gamma irradiation to permit the desired molecular weight reduction to occur (Miller, R. & Shiedlin, A. U.S. Pat. No. 6,383,344). In some embodiments, the hyaluronic acid is essentially water soluble. The hyaluronic acid can be a modified hyaluronic acid. For example, carboxyl group in the glucuronic acid portion of hyaluronic acid can be converted to a substituted amide group. Suitable substituents of the above substituted amide group may include: an aminoalkyl group (the alkylene chain of which may be substituted with one or more, namely, for example 1 to 8, and preferably 1 to 3 hydroxyl groups.); an amino(polyalkyleneoxy)alkyl group; an amino(polyalkyleneamino)aUcyl group; a mercapto(polyalkyleneamino)alkyl group; an acryloyloxyalkyl group; an acryloylaminoalkyl group; and an acryloylamino(polyalkyleneoxy)alkyl group.
Alternatively, the hyaluronic acid can be cross-linked by any method well known in the art (for example, WO2005085329).

The present invention also considers one or more salt(s) of hyaluronic acid. In cases where a hyaluronic acid salt is a salt with an inorganic base, such as alkali metal (e.g., lithium, sodium, or potassium) or alkaline earth metal (magnesium or calcium), such salts are easily obtained, for example, by reacting hyaluronic acid with a base containing an alkali metal or alkaline earth metal. The hyaluronic acid salt (e.g., silver salt) can also be prepared by methods as disclosed in U.S. Pat. Nos. 4,784,991 and 5,472,950. In addition, the hyaluronic acid salt can also be purchased from a variety of commercial sources.

In the invention, the hyaluronic acid, or one of its salts, is present in the sterile and injectable aqueous formulation at a concentration comprised between 5 to 45 mg/ml, most preferably between 8 to 40 mg/ml, more preferably between 8 to 30 mg/ml and even more preferably between 15 to 30 mg/ml.

In the invention, the hyaluronic acid, or one of its salts, has an average molecular weight equal or higher than 500 000 daltons e.g. equal or higher than 600 000 daltons, equal or higher than 700 000 daltons, equal or higher than 800 000 daltons, equal or higher than 900 000 daltons, equal or higher than 1 000 000 daltons, equal or higher than 2 000 000 daltons, or equal or higher than 3 000 000 daltons.

The hyaluronic acid, or one of its salts, present in the aqueous formulation of the invention can be either i) non-cross-linked or essentially non-cross-linked, or ii) cross-linked or essentially cross-linked.

In case the hyaluronic acid, or one of its salts, is non-cross-linked or essentially non-cross-linked, then its concentration is preferably comprised between 8 to 45 mg/ml.
In case the hyaluronic acid, or one of its salts, is cross-linked or essentially cross-linked, then its concentration is preferably comprised between 5 to 45 mg/ml.

The sterile and injectable aqueous formulation of the invention also includes at least one polyol. “Polyol(s)”, as used herein, refers to one or more compounds with multiple hydroxyl functional groups. Preferably, the at least one polyol is present in the sterile and injectable aqueous formulation at a concentration equal or higher than 7 mg/ml, most preferably at a concentration equal or higher than 15 mg/ml.

The Applicants of the present invention have shown that the efficacy of the rapid pain relief is improved with an increase of the polyol concentration. However, a polyol has a high capacity to increase the osmolarity of the formulation of the present invention. Consequently, the polyol concentration has to be adapted (maximum polyol concentration) for each formulation to obtain a product with a high polyol concentration equal or higher than 7 mg/ml with a physiological osmolarity in order to avoid side effects as joint effusion.

In accordance with a preferred embodiment of the invention the polyol is selected from the group comprising sorbitol, glycerol, mannitol, propylene glycol, xylitol, and/or combinations thereof. Most preferably, the polyol is the sorbitol.

As known in the art, the presence of a polyol in a sterile aqueous formulation that is based on hyaluronic acid makes it possible to increase significantly the resistance to the degradation of this gel. A strong affinity of the hyaluronic acid and the polyol within the sterile gel involves a slow kinetics for release of the polyol outside of the gel: this affinity between the hyaluronic acid and the polyol involves an effective long-term protection of the gel by the polyol by a synergetic action.

Surprisingly, the Applicants of the present invention have shown that the presence of polyol not only increases significantly the resistance to the degradation of the formulation of the invention but also has a rapid and strong beneficial effect on pain (rapid reduction of the pain similar to the pain relief obtained with a corticosteroid injection) when combined to hyaluronic acid in accordance with the invention. These properties have never been reported before and were thus unexpected due to the non pharmacological nature of the polyol, which plays a key role in the formulation of the present invention.

Additionally, the use of a polyol instead of a corticosteroid avoids the appearance of side effects associated with said corticosteroid such as post-injection flare, synovitis and potential cartilage damage with repeated use.

The above-mentioned properties are observed when the ratio between the concentrations of the at least one polyol and acid hyaluronic, or one of its salts, is comprised between about 0.155 to 14 and when said formulation has a zero-shear viscosity η0 equal or higher than 15 Pa·s.

The “zero-shear viscosity η0” refers to the viscosity which is measured at low shear rate, close to 0 s-1.

The zero-shear rate viscosity η0 of the sterile and injectable aqueous formulation of the invention is equal or higher than 15 Pa·s, preferably equal or higher than 50 Pa·s, more preferably equal or higher than 100 Pa·s and even more preferably equal or higher than 200 Pa·s.

The Applicants of the present invention have also shown that the above-mentioned properties are observed when the ratio between the concentrations of the at least one polyol and acid hyaluronic, or one of its salts, is comprised between about 0.155 to 14.

Pain relief is inexistent if the ration is below 0.155 whereas there are side effects when the ratio is above 14.

While not wishing to be bound by any theory as to mechanism of action, it is believed that the polyol, in synergy with the hyaluronic acid and in the specific conditions of the invention, provides a beneficial effect on inflammation by reducing said inflammation and thus enabling a rapid and strong pain relief.

While referring to the examples, pain reduction is much more important when a formulation according to the invention (formulation H1) is used compared to the reduction obtained with one injection of a viscosupplement based on hyaluronic acid (Formulation H2), when assessed after one or three weeks post treatment. In contrast, Formulations C and G (examples 1 and 2), where the concentrations of polyol are less than 7 mg/ml, do not show a beneficial effect on inflammation in comparison to a formulation of reference.
It also appears clearly that the zero-shear rate viscosity η0 of the sterile and injectable aqueous formulation of the invention must be equal or higher than 15 Pa·s in order to achieve inflammation and pain reductions. Formulation D, as depicted in the examples, promotes only 9% inflammation reduction which is not significantly different from the effect promoted by the formulation of reference.

The hydrogel according to the invention can contain various common additives or pharmacologically active agents or other ingredients beneficial for the formulation or for the treated subject. These common additives, pharmacologically active agents and other ingredients are known in the art.

According to the invention, the formulation is sterile. Sterilization is performed by the methods well known in the art. Preferably, the formulation of the invention is sterilized by autoclave.

The sterile and injectable aqueous formulation of the invention is adapted for the administration in one single dose injection. This treatment regimen (only one injection to obtain a rapid and strong pain relief) is absolutely necessary to have the possibility to have a therapeutic solution which can be competitive with the treatment using a corticosteroid.

The volume to be injected in a single dose is dependent of the size of the joint which has to be treated. Typically, the volume of injection is comprised between 0.1 ml (for the smallest joints) and 10 ml (for the biggest joints).

In order to maximize the rapid and strong pain relief, the volume of injection has to be adapted to the size of the joint and to the exact formulation according to the invention (concentration of hyaluronic acid, molecular weight of the hyaluronic acid, polyol and concentration of the polyol, zero-shear viscosity of the hydrogel, . . . ).

However, the volume to be administrated must not be too important. If it is the case, it induces a physical destabilization of the joint and side effects.

Typically, in human medicine, the volume is about 3 to 8 ml for the knee, about 1.5 to 4 ml for the hip, about 1.5 to 4 ml for the ankle, about 1.5 to 4 ml for the shoulder.

In accordance with various embodiments of the present invention, the sterile and injectable aqueous formulation is for use in the treatment and/or prevention of pain associated with an intra-articular joint degeneration or disease.

Also envisioned in the present invention is the use of the sterile and injectable aqueous formulation described herein in the treatment and/or prevention of cartilage destruction in the intra-articular joint of a subject.

The present invention also considers a method of treatment and/or prevention of cartilage destruction, or of pain associated with an intra-articular joint degeneration or disease, comprising the injection in one single dose in a subject, of a sterile and injectable aqueous formulation in accordance with the invention.

Cartilage destruction and intra-articular joint degeneration are observed, for example in Rheumatoid arthritis and Osteoarthritis.

Rheumatoid arthritis refers to a chronic systemic autoimmune inflammatory disease that mainly involves the synovial membrane of multiple joints with resultant injury to the articular cartilage, resulting in joint destruction. The main presenting symptoms in rheumatoid arthritis are pain, stiffness, swelling, and/or loss of function of one or more joints.

Osteoarthritis is characterized by the degeneration of articular cartilage, changes of the subchondral bone, associated pain, and increasing disability of the affected joint. Osteoarthritis most commonly affects joints of the hands, hips, knees, and spine. Osteoarthritis of the knee joint is particularly common.

The present invention also provides a medical device comprising a sterile and injectable aqueous formulation of the invention for use in the treatment and/or prevention of pain associated with an intra-articular joint degeneration or disease. For example, the medical device is a syringe comprising a composition described herein according to any embodiment.

A kit comprising a sterile and injectable aqueous formulation of the invention, or the device of the invention, for use in the treatment and/or prevention of pain associated with an intra-articular joint degeneration or disease is also contemplated.

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

### EXAMPLES

### Example 1

### Preparation of Formulations

The formulations prepared in the following examples are gels based on non-cross-linked or cross-linked sodium hyaluronate (NaHA) containing a polyol in an aqueous solution buffered at pH=7.

The preparation of hydrogels proposed in these examples is performed using techniques well known to those skilled in the art. In the case of the hydrogel based on cross-linked NaHA, the crosslinking agent used is butanediol diglycidyl ether (BDDE) and the definition of the crosslinking rate in that case is: mass (BDDE)/mass (dry NaHA).
The incorporation of polyol in the gel is made by adding the required amount of polyol in the non-cross-linked or cross-linked gel and mixing during 10 minutes with a spatula (for a final amount of gel of 50 g).
All the studied hydrogels are manufactured under clean conditions (control of bioburden during manufacturing).
In the last step of manufacturing, all the hydrogels are packaged in glass syringe and some of these hydrogels are sterilized under moist heat according to the conditions described for each formulation.
The rheometer used for the measurement of zero-shear viscosity is a AR2000 (TA Instruments) with a flat geometry of 40 mm, a gap of 1000 μm and an analysis temperature of 25° C.

### Formulation A: (According to the Invention)

Hydrogel based on non-cross-linked sodium hyaluronate and sorbitol

8 mg/ml of NaHA at 8×10⁵ Da

7 mg/ml of sorbitol

Phosphate buffer to a final volume of 1 ml

Hydrogel packaged in glass syringe and sterilized under moist heat at 121° C. during 20 minutes.
The zero-shear viscosity of the Hydrogel is 17 Pa·s.

### Formulation B: (According to the Invention)

Hydrogel based on non-cross-linked sodium hyaluronate and sorbitol
15 mg/ml of NaHA at 2.8×10⁶ Da
25 mg/ml of sorbitol
Phosphate buffer to a final volume of 1 ml
Hydrogel packaged in glass syringe and sterilized under moist heat at 121° C. during 20 minutes.
The zero-shear viscosity of the Hydrogel is 193 Pa·s.

### Formulation C:

Hydrogel based on non-cross-linked sodium hyaluronate and sorbitol
8 mg/ml of NaHA at 8×10⁵ Da
5 mg/ml of sorbitol
Phosphate buffer to a final volume of 1 ml
Hydrogel packaged in glass syringe and sterilized under moist heat at 121° C. during 20 minutes.
The zero-shear viscosity of the Hydrogel is 15 Pa·s.

### Formulation D:

Hydrogel based on non-cross-linked sodium hyaluronate and sorbitol
8 mg/ml of NaHA at 6×10⁵ Da
15 mg/ml of sorbitol
Phosphate buffer to a final volume of 1 ml
Hydrogel packaged in glass syringe and sterilized under moist heat at 121° C. during 20 minutes.
The zero-shear viscosity of the Hydrogel is 5 Pa·s.

### Formulation E: (According to the Invention)

Hydrogel based on cross-linked sodium hyaluronate and sorbitol
5 mg/ml of NaHA at 2.8×10⁶ Da, crosslinking rate=4%
7 mg/ml of sorbitol
Phosphate buffer to a final volume of 1 ml
Hydrogel packaged in glass syringe and sterilized under moist heat at 121° C. during 20 minutes.
The zero-shear viscosity of the Hydrogel is 42 Pa·s.

### Formulation F: (According to the Invention)

Hydrogel based on non cross-linked sodium hyaluronate and glycerol
30 mg/ml of NaHA at 6×10⁵ Da
7 mg/ml of glycerol
Phosphate buffer to a final volume of 1 ml
The zero-shear viscosity of the Hydrogel is 21 Pa·s.

### Formulation G:

Hydrogel based on non cross-linked sodium hyaluronate and glycerol
30 mg/ml of NaHA at 6×10⁵ Da
5 mg/ml of glycerol
Phosphate buffer to a final volume of 1 ml
The zero-shear viscosity of the Hydrogel is 20 Pa·s.

### Control Solution:

Phosphate buffer solution containing sorbitol at 25 mg/ml
Solution sterilized under moist heat at 121° C. during 20 minutes.

### Example 2

### In Vivo Evaluation of Different Formulations

An in vivo evaluation of formulations A to G is performed on rats by inducing inflammation in joints, then treating joints with formulations A to G, and analyzing inflammation by 2D bioluminescence measurements.
For each joint, inflammation is induced by injection of 25 microliters of a carrageenan solution at 1% in physiological buffer. 24 hours later, each joint is treated with 200 microliters of a formulation. For each formulation, measurement of the inflammation is performed on 4 different joints, 5 hours following injection of the carrageenan solution and 1 day following injection of the test formulation.

### Results

| | Inflammation decrease |
|---|---|
| | Ratio of inflammation measured at 1 day following |
| | injection of the test formulation against inflammation |
| | at 5 hours following injection of the carrageenan |
| Formulation | solution (Mean on 4 articulations) |
| A | −26% |
| B | −78% |
| C | −8% |
| D | −9% |
| E | −19% |
| F | −24% |
| G | −6% |
| Control | −8% |

The formulations according to the invention reduce significantly the level of inflammation within joints

### Example 3

### Clinical Evaluation

The formulation H1 according to the invention is as follows:
Hydrogel based on non-crosslinked sodium hyaluronate and sorbitol

20 mg/ml of NaHA at 2.6×10⁶ Da

40 mg/ml of sorbitol

Phosphate buffer to a final volume of 1 ml

Hydrogel packaged in glass syringe and sterilized under moist heat at 121° C. during 20 minutes.
The zero-shear viscosity of the Hydrogel is 375 Pa·s.
The formulation H2 according to the prior art is a marketed viscosupplement formulation consisting of:

20 mg/ml NaHA at 1.8×10⁶ Da

Phosphate buffer to a final volume of 1 ml

Hydrogel packaged in glass syringe and sterilized under moist heat
The zero-shear viscosity of the Hydrogel is 148 Pa·s.
A clinical study was conducted by a single practitioner to evaluate the effect of formulations H1 and H2 on the knee joints of 6 patients with Osteoarthritis (OA).
Patients were randomized as follows:
3 patients received a single dose injection of 4 ml of hydrogel H1, 3 patients received a single dose injection of 4 ml of hydrogel H2.
There was one joint treated per patient.
The pain during walking was assessed for each patient before injection, 1 week after injection and 3 weeks after injection.

### Results

| Decrease of the Pain during | | |
|---|---|---|
| walking: Ratio of the Pain | | |
| measured with treated patients | | |
| against Pain measured during | Formulation H1 | |
| their non-treated condition | (according to the | Formulation H2 |
| (Mean on 3 articulations) | invention) | (prior art) |
| At 1 week following treatment | −59% | −18% |
| At 3 weeks following | −67% | −27% |
| treatment | | |

1. A sterile and injectable aqueous formulation for administration in the intra-articular space of an intra-articular joint of a subject, in the form of a gel comprising:
i) hyaluronic acid, or one of its salts, at a concentration comprised between 5 to 45 mg/ml, said hyaluronic acid, or one of its salts, having an average molecular weight equal or higher than 500,000 daltons, and
ii) a polyol at a concentration equal or higher than 7 mg/ml,
wherein the ratio between the concentrations of polyol and acid hyaluronic, or one of its salts, is comprised between about 0.155 to 14, and wherein said sterile and injectable aqueous formulation is adapted for the administration in one single dose injection and has a zero-shear rate viscosity η0 equal or higher than 15 Pa·s.

2. The sterile and injectable aqueous formulation of claim 1, wherein the intra-articular space is selected from the group consisting of the intra articular joint of the ankle, wrist, hand joint, knee, hip, foot joint, spine joint, shoulder joint, and any other joint or space.

3. The sterile and injectable aqueous formulation of claim 1, wherein the hyaluronic acid, or one of its salts, is non-cross-linked or essentially non-cross-linked.

4. The sterile and injectable aqueous formulation of claim 3, wherein the concentration of hyaluronic acid, or of one of its salts, is comprised between 8 to 45 mg/ml.

5. The sterile and injectable aqueous formulation of claim 1, wherein the hyaluronic acid, or one of its salts, is cross-linked or essentially cross-linked.

6. The sterile and injectable aqueous formulation of claim 5, wherein the concentration of hyaluronic acid, or of one of its salts, is comprised between 5 to 45 mg/ml.

7. The sterile and injectable aqueous formulation of claim 1, wherein volume of the single dose injection is comprised between 0.1 ml and 10 ml.

8. The sterile and injectable formulation of claim 1 for use in the treatment and/or prevention of pain associated with an intra-articular joint degeneration or disease.

9. A device comprising a sterile and injectable aqueous formulation of claim 1 for use in the treatment and/or prevention of pain associated with an intra-articular joint degeneration or disease.

10. A kit comprising a sterile and injectable aqueous formulation of claim 1, for use in the treatment and/or prevention of pain associated with an intra-articular joint degeneration or disease.

11. A method of treatment and/or prevention of pain associated with an intra-articular joint degeneration or disease comprising the injection in one single dose in a subject, of a sterile and injectable aqueous formulation, in the form of a gel, said sterile and injectable aqueous formulation comprising:
i) hyaluronic acid, or one of its salts, at a concentration comprised between 5 to 45 mg/ml, said hyaluronic acid, or one of its salts, having an average molecular weight equal or higher than 500,000 daltons, and
ii) a polyol at a concentration equal or higher than 7 mg/ml,
wherein the ratio between the concentrations of polyol and acid hyaluronic, or one of its salts, is comprised between about 0.155 to 14, and wherein said sterile and injectable aqueous formulation is adapted for the administration in one single dose injection and has a zero-shear rate viscosity η0 equal or higher than 15 Pa·s.
